(19) United States
(12) Patent Application Publication
(10) Pub. No.: US 2008/0208009 A1
(43) Pub. Date: Aug. 28, 2008

(54) Wearable Device, System and Method for Measuring Vital Parameters

(76) Inventor: Dror Shklarski, Yavne (IL)

Correspondence Address: EMPK & Shiloh, LLP, 116 JOHN ST,, SUITE 1201, NEW YORK, NY, 10038, US

(21) Appl. No.: 11/631,947

(22) PCT Filed: Jul. 10, 2005
(86) PCT No.: PCT/IL05/00732

§ 371 Date: Dec. 26, 2007

Related U.S. Application Data
(60) Provisional application No. 60/586,241, filed on Jul. 9, 2004.

(51) Int. Cl. A61B 5/1455

(52) U.S. Cl. 600/301; 600/324

(57) ABSTRACT

A wireless mobile device is provided for measuring pulse and blood oxygen saturation (SpO2). The device may include a SpO2 sensor, a pulse sensor, and a main controller to receive and process signals from the SpO2 and the Pulse sensors, and to enable reconfiguration of the SpO2 and the Pulse sensors by commands received from a remote server. The device may include a light measurement module to measure pulse parameters, and a light measurement module to measure SpO2 parameters, the light measurement modules including an emitting/receiving unit and an electronic unit.

### FIELD OF THE INVENTION

The present invention relates to wearable devices, systems and methods for monitoring and evaluating physiological and/or environmental parameters, and particularly, to devices, systems and methods for measuring pulse and blood oxygen saturation levels.

### BACKGROUND OF THE INVENTION

Continuously monitoring a patient's physiological condition generally requires the patient's hospitalization, usually at great cost, especially where long term monitoring is required. In certain situations it is possible to monitor the physiology of patients who are physically outside of the hospital, using wearable monitoring devices.

There are, for example, wrist-worn devices that may record a patient's physiological data, such as the patient's ECG, during a predetermined recording time. These devices may include event recorders that may capture a patient's physiological data during a physiological “event”, such as a cardiac arrhythmia or an episode of patient discomfort. The event recording may be activated manually by the patient or automatically by determining when monitored physiological data meets predefined event criteria.

Wrist-worn devices typically may require that a patient return to a medical center periodically or remotely communicate with a medical center in order to transfer the recorded data for interpretation by a medical staff.

### SUMMARY OF THE INVENTION

A wireless mobile device is provided for measuring pulse and blood oxygen saturation (SpO₂). The device may include a SpO₂ sensor, a pulse sensor, and a main controller to receive and process signals from the SpO₂ and the Pulse sensors, and to enable reconfiguration of the SpO₂ and the Pulse sensors by commands received from a remote server. The device may include a light measurement module to measure pulse parameters, and a light measurement module to measure SpO₂ parameters, the light measurement modules including an emitting/receiving unit and an electronic unit.

According to some embodiments of the present invention, a system is provided for measuring pulse and SpO₂, comprising a wireless mobile monitoring device including an array of sensors; and a medical center server enabled to remotely initiate measurements of pulse and SpO₂ levels of a patient using the monitoring device, wherein the mobile monitoring device includes a light measurement module, the light measurement model having an emitting/receiving unit and an electronic unit.

According to some embodiments of the present invention, a method is provided for measuring pulse and SpO₂, the method comprising transmitting commands to a wireless monitoring device, from a medical center server, to remotely measure pulse and SpO₂ of a patient using the monitoring device, wherein the mobile monitoring device includes a light measurement module to measure pulse parameters, and a light measurement module to measure SpO₂ parameters.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIG. 1 is a schematic illustration of a medical monitoring system according to some exemplary embodiments of the present invention;

FIGS. 2A, 2B, and 2C are schematic illustrations of external top, bottom, and side view layouts, respectively, of a wearable monitoring device according to some exemplary embodiments of the present invention;

FIGS. 3A and 3B are schematic illustrations of a SpO₂ sensor and a pulse sensor, respectively, according to some exemplary embodiments of the present invention;

FIGS. 4A-4I are schematic illustrations of nine sensor configurations incorporating SpO₂ sensors and pulse sensors, according various exemplary embodiments of the present invention; and

FIG. 5 is a schematic illustration of an internal layout of a wearable monitoring device, according to some exemplary embodiments of the present invention.

### DETAILED DESCRIPTION OF THE INVENTION

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

In the following description, various aspects of the invention will be described. For purposes of explanation, specific configurations and details are set forth in order to provide a thorough understanding of the invention. However, it will also be apparent to one skilled in the art that the invention may be practiced without the specific details presented herein. Furthermore, well-known features may be omitted or simplified in order not to obscure the invention.

Unless specifically stated otherwise, as apparent from the following discussions, it is appreciated that throughout the specification discussions utilizing terms such as “processing,” “computing,” “calculating,” “determining,” or the like, refer to the action and/or processes of a computer or computing system, or to a similar electronic computing device, that manipulates and/or transforms data represented as physical, such as electronic quantities within the computing system's registers and/or memories into other data similarly represented as physical quantities within the computing system's memories, registers or other such information storage, transmission or display devices.

The processes and displays presented herein are not inherently related to any particular apparatus. Various general-purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of embodiments of the invention as described herein.

It should be appreciated that according to some embodiments of the present invention, the method described below may be implemented in machine-executable instructions. These instructions may be used to cause a general-purpose or special-purpose processor that is programmed with the instructions to perform the operations described. Alternatively, the operations may be performed by specific hardware that may contain hardwired logic for performing the operations, or by any combination of programmed computer components and custom hardware components.

Although the scope of the present invention is not limited in this respect, the wearable device disclosed herein may be implemented in any suitable wired or wireless device that may be a handheld, worn, or other suitable portable communications device. By way of example, the wearable devices may include wireless and/or cellular telephones, smart telephones, personal digital assistants (PDAs), wrist-worn devices, and other suitable wearable devices or any parts of them. Alternatively, according to other embodiments of the present invention, the system and method disclosed herein may be implemented in computers.

Embodiments of the present invention are directed to an improved wearable device, a system, and a method for monitoring medically relevant parameters and/or processing data related to medically relevant parameters and/or alerting a patient and/or medical facility regarding a medical condition. For example, devices, systems and methods may be provided for monitoring physiological and/or environmental parameters, sending alerts to a Medical Center (MC) and/or to a patient, processing sensed data, and updating device parameters and/or functions and/or its software. The wearable device, according to some embodiments of the present invention, may enable measurement of pulse and blood oxygen saturation (SpO₂) from one or more locations in or on a user's body. In other embodiments, additional physiological and/or environmental parameters, or combinations of parameters may be measured.

The wearable device, according to some embodiments, may independently transfer a patient's physiological and/or environmental data, and/or other suitable data to, for example, the MC. For example, when certain parameters are above or below predetermined ranges or thresholds that may be defined according to the particular needs of a patient, such data and/or signals or messages associated with the data may be sent to the MC. In some embodiments, the wearable device may also transfer the data to the MC if the parameters are within predetermined thresholds or ranges. In some embodiments, the MC may receive, via a communications channel, a patient's physiological data and additional information, such as, for example, the location of the patient, directly from the wearable device. A person or persons associated with the MC may remotely update, for example, the ranges or thresholds for determining the status of vital parameters of an individual patient, at the discretion of the staff, for example, medical staff, information technology staff and/or technical/engineering staff in the MC, or a device associated with the MC's server, may automatically update various parameters. Additionally, the MC server may remotely update operational features, for example, changing modes of operation, adding new features, updating the device's software and/or part thereof etc. for a wireless device of an individual patient or for a group of patients. Any wearable or remote device for monitoring, measuring, communicating etc. vital parameter or other data of a patient to the MC may be used.

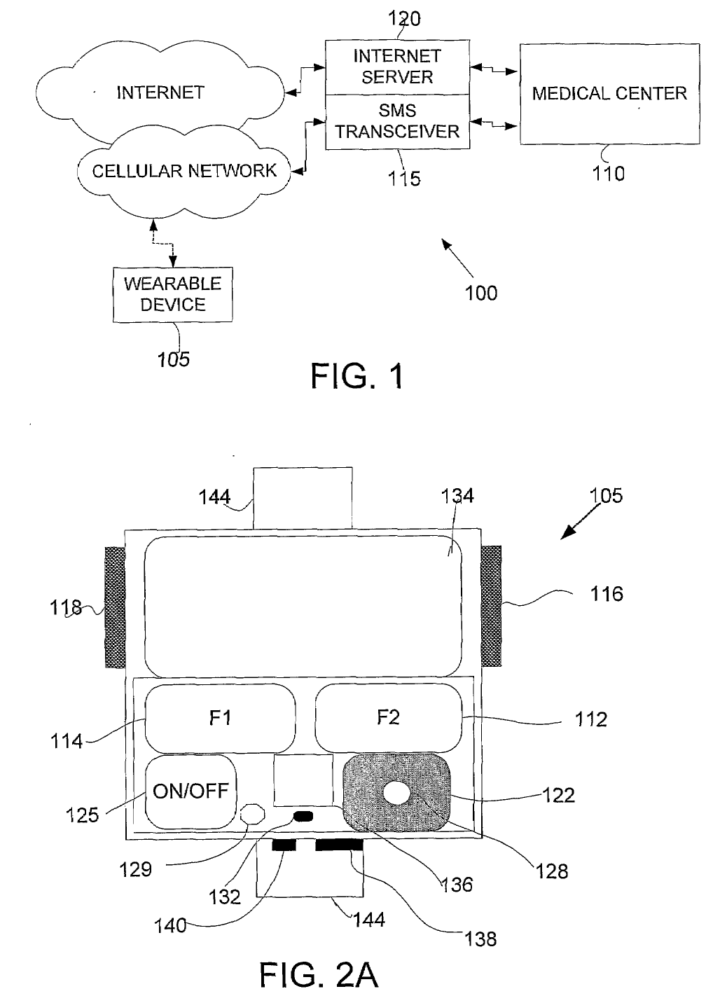

Reference is now made to FIG. 1, which schematically illustrates a medical monitoring system 100 in accordance with some exemplary embodiments of the present invention. Medical monitoring system 100 may include, for example, at least one wearable device 105 that may communicate with a medical center (MC) server 110. Wearable device 105 may have a bi-directional communication link with MC server 110, which may be associated with, for example, a clinic, hospital, remote center, medical professional, or any other suitable provider of suitable medical services. For example, wearable device 105 may communicate with MC server 110 using a serial communication port, a parallel connection, USB, a modem, network card (e.g., ADSL, Cable, satellite) or other data communications technologies. For example, wearable device 105 may communicate with MC server 110 using wireless data communication, for example, using cellular communication (e.g., General Packet Radio Service (GPRS)), satellite communications technology, wireless LAN technology, infrared technology, Wireless Fidelity (WiFi), Bluetooth, ZigBee, or other suitable wireless communications technologies. Data may be transferred between wearable device 105 and MC server 110 using the above or other suitable means.

The communication may be performed over a computer network, for example, the Internet or a local area network (LAN), etc. There may be a plurality of bi-directional and/or uni-directional communication channels between MC server 110 and wearable device 105, and there may be a plurality of medical centers (MC), MC servers 110 and/or wearable devices 105.

In one embodiment the bi-directional communication channel between the MC server 110 and wearable device 105 is a Short Message Service (SMS) channel that may enable communication of data via SMS transceiver 115 to and/or from the wearable device 105, via a cellular communications network. The SMS channel may enable transmission of messages from wearable device 105 to MC server 110, via SMS transceiver 115. In one embodiment the bi-directional communication channel between the MC server 110 and wearable device 105 is an Internet Protocol (IP) based channel, that may enable communication of data via Internet server 120, for example, using File Transfer Protocol (FTP) or other suitable data transfer protocols. In some embodiments a combination of communication networks may be used. For example, if the SMS channel is not available and/or not chosen by the wearable device 105, wearable device 105 may communicate with MC server 110 using FTP. In other embodiments wearable device 105 may communicate with MC server 110 using, for example, SMS and Internet communications. In some embodiments wearable device 105 may communicate with MC server 110, via a Web interface, for example, a Website, where data, commands, and/or requests etc. may be entered and/or received by wearable device 105 and/or MC server 110.

In one embodiment the bi-directional communication channel between the MC server 110 and wearable device 105 may utilize TCP/IP protocol. In one embodiment a File Transfer Protocol (FTP) may be used to upload physiological data of the patient, e.g., sensed measurement data, from wearable device 105 to MC server 110, and to download data such as updates to software modules from MC server 110 to wearable device 105. Usage of FTP or any other suitable protocol may require the wearable device 105 to logon as an FTP client to the Internet server 120.

In some embodiments a voice channel, as described below, may be used to enable the staff at MC server 110, or a device or suitable software and/or hardware associated with the MC server 100, to communicate with the patient who is using wearable device 105 and/or to enable the patient using wearable device 105 to communicate with the staff of MC server 110 or the MC server 110 itself.

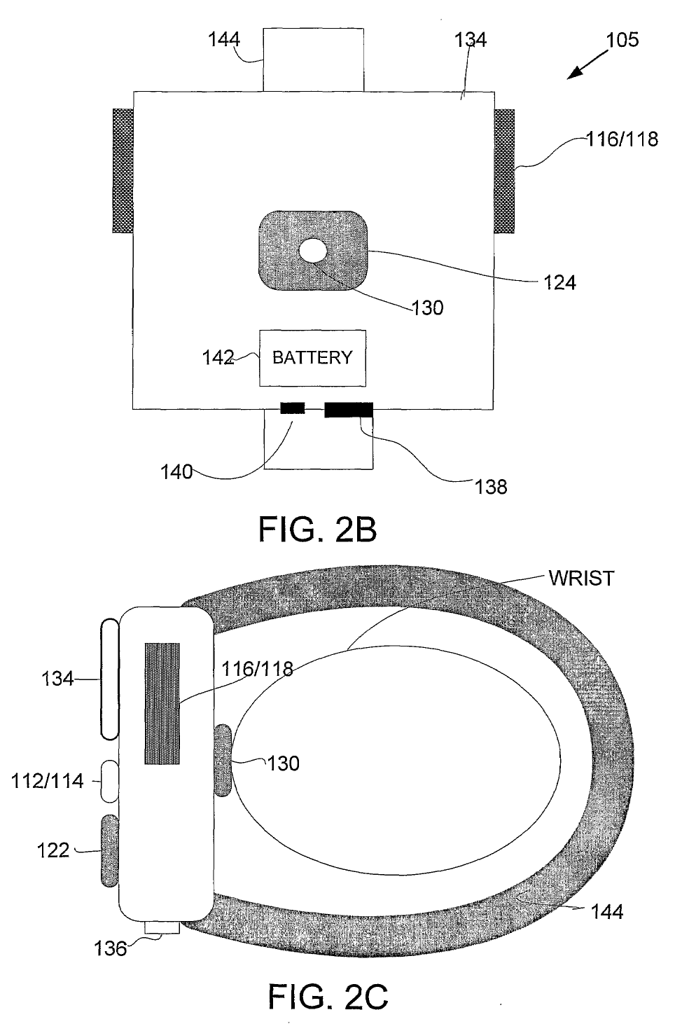

Reference is now made to FIGS. 2A, 2B, and 2C, which schematically illustrate examples of external top, bottom, and side view layouts, respectively, of a wearable device 105 in accordance with some exemplary embodiments of the present invention. Wearable device 105 may include, for example, input components such as functional buttons 112 and 114 for inputting data or commands (e.g., approving or rejecting activities) to operate wearable device 105, emergency buttons 116 and 118, that may be used to manually initiate an emergency mode (e.g., by pressing them together or pressing one of them), and an On/Off button 125 to switch wearable device 105 on or off. The on/Off button 125 may be unified with any of the other buttons, for example functional buttons 112 and 114. Wearable device 105 may include one or more electrodes, for example, an ECG RA (Right Arm) finger electrode 122, an ECG LA (Left Arm) wrist electrode 124 (FIG. 2B), and an ECG REF. (Reference) wrist electrode 126 (shown in FIG. 2C). Electrodes 122 and 124 may be located at any suitable location or locations on wearable device 105. For example, electrode 124 may be located on the top-side of wearable device 105. In some embodiments, the ECG REF. Wrist electrode 126 may be located at any suitable location on the inner side of wearable device 105 or on the inner side of a strap 144. Wearable device 105 may be worn on a patient's left or right hand or arm, e.g., on the wrist, or on the left or right foot or leg, e.g., on the ankle, and the various components may be appropriately located to enable measuring of parameters on the left and/or right hand and/or arm and/or foot and/or leg.

In some embodiments wearable device 105 may include at least one blood oxygen level (SpO₂) transceiver 128 to measure the level of the oxygen in the patient's blood, and at least one pulse transceiver 130 (shown in FIG. 2B) to measure the patient's pulse. Blood oxygen saturation level (SpO₂) transceiver 128 may be incorporated into, for example, electrode 122 and/or may be independent of electrode 122. In some embodiments wearable device 105 may include a pulse transceiver or sensor 130. The location of pulse transceiver 130 within wearable device 105 may be appropriately positioned to enable sensing of the pulse of the patient. Pulse transceiver 130 may be incorporated into, for example, electrode 124 and/or may be independent of electrode 124. In some embodiments, wearable device 105 may include one or more transceivers, electrodes, or sensors to enable measurement of SpO₂, ECG, pulse, blood pressure data, skin temperature data, respiration data, perspiration data, cardio impedance data, blood sugar or glucose level, and/or other suitable data. The SpO₂, pulse and/or other parameters may be presented on a display area 134 of wearable device 105. The pulse and/or other parameters may be transferred to the MC server 110. Other sensor mechanisms may be used.

Wearable device 105 may include a speaker 136 to enable a patient to hear audio signals, for example from voice communications initiated from MC server 110 or from other sources. When wearable device 105 is operated in a continuous mode of operation, wearable device 105 may, for example, continuously or according to a pre-defined schedule, read the pulse of the patient, using pulse transceiver 130. The location of pulse transceiver 130 within wearable device 105 may be appropriately positioned to enable sensing of the pulse of the patient. Pulse transceiver 130 may be incorporated within electrode 124 or may be separate from electrode 124. An indication of the pulse of the patient and/or other parameters may be presented on the display area 134 of wearable device 105. The pulse and/or other parameters may also be transferred to the MC server 110. Other sensor mechanisms may be used.

Display area 134 may display additional information such as, for example, medical parameters of the patient, messages received from MC server 110, operational instructions, date and time, parameters that are related to functional elements of wearable device 105 etc. Display area 134 may be, for example, a color display and/or a monochromatic display and may have any desired resolution, depending on the type of data to be displayed. In some embodiments, display area 134 may include an interactive display, for example, a touch sensitive display, and may have a voice-activated circuit to control Display area 134. Display area 134 may display any combination of alphanumeric characters, and/or text and/or two-dimensional and/or three-dimensional graphics and/or icons.

Additional elements in wearable device 105 may include one or more service connectors, for example, a service connector 138 that may connect the wearable device 105 to external units such as, for example, a computer that may help provide software updates, testing, technical diagnostics etc., a testing unit that may enable testing the usability of device 105, an external medical device, for example, To measure blood pressure, ECG etc., an external display unit, communication unit, for example, a Bluetooth chip and circuitry, and/or other suitable external units. Wearable device 105 may include a charge connector 140 that may be used to connect wearable device 105 to a power source to enable charging of a battery 142 (FIG. 2B). A charger connector 140 may be included in service connector 138. Wearable device 105 may include optional strap 144 that may be used to attach wearable device 105 to the wrist or other location of the patient. Wearable device 105 may include various other suitable components and/or devices, which may be implemented using any suitable combination of elements and components and may incorporate hardware and/or software.

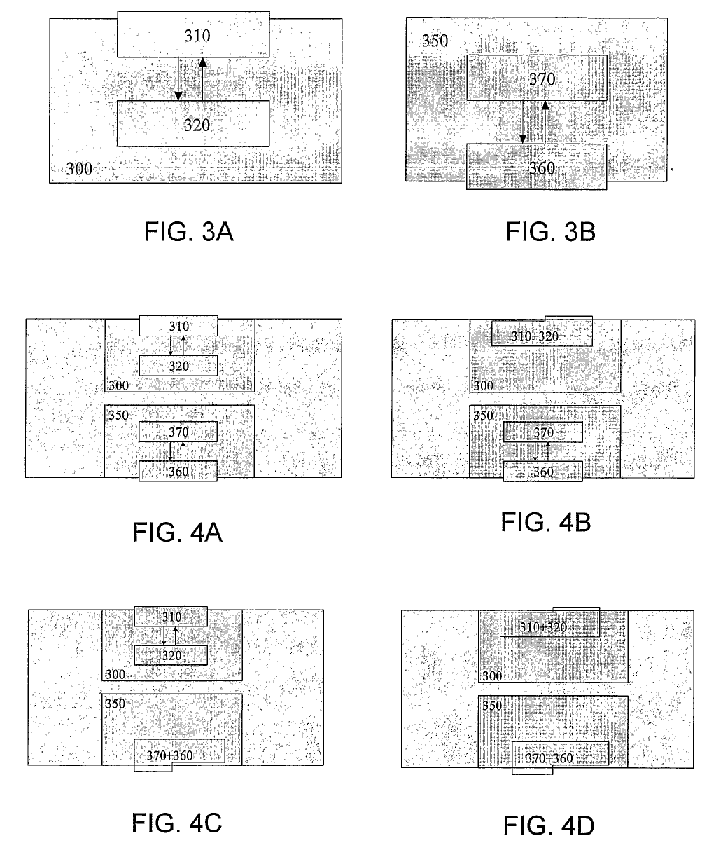

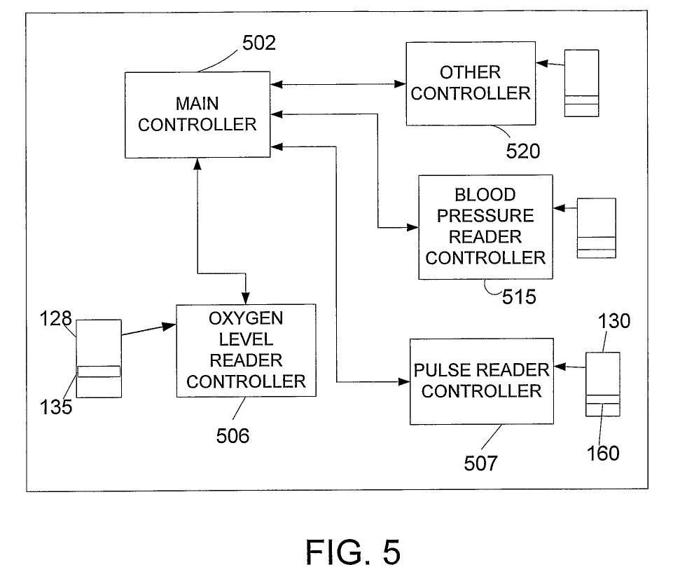

Reference is now made to FIGS. 3A and 3B, which schematically illustrate possible sensor configurations to perform the functions described above with reference to SpO₂ sensor 128 (FIG. 2A) and pulse sensors 130 (FIG. 2B), respectively. According to some embodiments of the present invention, wearable device 105 may have one or more SpO₂ sensors 300 (FIG. 3A) and/or one or more pulse sensors 350 (FIG. 3B). Each SpO₂ sensor 300 may include at least one emitting/receiving unit 310 and at least one electronic unit 320 as well as other appropriate components. Emitting/receiving unit 310 may be located, for example, on the upper side of wearable device 105, e.g., to be reachable by at least one finger of the patient wearing the device 105, or in the inner side of strap 144. Electronics unit 320 may be connected to a main controller (e.g., controller 502 in FIG. 5 below) of wearable device 105, and may optionally receive power from battery 142 (FIG. 2B) or from a battery circuit. SpO₂ sensor 300 may enable measurement of blood oxygen saturation in the blood and/or a patient's pulse. Each pulse sensor 350 may include at least one emitting/receiving unit 360 and at least one electronic unit 370 as well as any other suitable components. Emitting/receiving unit 360 may be located, for example, on the lower side of wearable device 105, e.g., to be in suitable contact with the wrist of the patient wearing the device, or on the upper side of wearable device 105. Electronics unit 370 may be connected to the main controller (see 502 in FIG. 5) of wearable device 105, and may optionally receive power from battery 142 or from the battery circuit. Measurements may be taken from each sensor at any time, and may be taken in series, in parallel, in response to a predefined trigger, continuously and/or according to other selected patterns. Sensors 300 and 350 may be located at other suitable positions. Other sensor types may be used in addition to or in place of sensors 300 and 350, for example, a blood pressure sensor SpA₂.

In accordance with some embodiments of the present invention, medical monitoring system 100 may operate in at least one of a keeper mode, an extended mode, and an emergency mode, or any other appropriate mode, as described below.

The keeper mode may be used as the default mode of wearable device 105, such that wearable device 105 may enter this mode when the device is switched on. Other modes may alternatively be used as the default mode. In the keeper mode, wearable device 105 may, for example, continuously or intermittently, read the pulse and/or another parameters of a patient. In one example of keeper mode functioning, wearable device 105 may display parameter data on display area 134, may alert the patient with a message on display area 134, and/or may alert the patient using an audible signal via speaker 136, for example, by playing back predefined audio signals. In addition, wearable device 105 may transmit the measured parameters to MC server 110 for analyses or processing of the measured parameters, for example, using a FTP channel and/or a SMS channel. In the event where the staff in MC server 110 determines that the patient's pulse is abnormal, according to predetermined criteria or ranges described in detail below, wearable device 105 may alert the patient.

According to some embodiments of the present invention wearable device 105 itself may determine when one or more parameters are abnormal or, for example, in a danger range, instead of or in addition to the staff in MC server 110. According to some embodiments of the present invention MC server 110 may automatically determine when one or more parameters are abnormal or, for example, in a danger range, instead of or in addition to the staff in MC server 110. Additionally, wearable device 105 may send a warning message to MC server 110, using, for example, the SMS channel, FTP channel etc. When wearable device 105 is operated in keeper mode, parameters such as pulse, SpO₂, and ECG may be monitored continuously and/or at selected intervals, for example, every twelve hours.

In the extended mode, wearable device 105 may be set to perform operations according to a pre-defined schedule, for example, to periodically measure oxygen levels in the patient's blood (SpO₂) and/or ECG. In this mode, wearable device 105 may display parameter data on display area 134, may alert the patient with a message on display area 134, and/or may alert the patient using an audible signal via speaker 136, for example, by playing back predefined audio signals. In addition, wearable device 105 may transmit the measured parameters and/or results from analyses or processing of the measured parameters, to MC server 110, for example, using FTP channel and/or SMS channel. When wearable device 105 is operated in extended mode, vital signs such as pulse, SpO2, and ECG, may be monitored, for example, five times a day by default (e.g., the default may be at shorter or longer intervals, as required). If the staff at MC server 110 or the MC server 110 detect, for example, that the heart rate, oxygen level in the blood, and/or ECG records and/or other data are abnormal (e.g., according to pre-defined criteria or ranges as discussed below), wearable device 105 may alert the patient by providing output signals in the display area 134 or via speaker 136. Additionally or alternatively, wearable device 105 may send a message to MC server 110, or to another destination, for example, using the FTP channel.

In emergency mode a patient may initiate operation of the medical monitoring system 100 by pressing, for example, any of the emergency buttons 116 or 118. When operating in emergency mode, wearable device 105 may send emergency messages to MC server 110 or to another destination using, for example, the FTP channel. Emergency messages may additionally or alternatively be sent to MC server 110 or to another destination via the SMS channel, for example, in cases where the FTP channel is not available. In addition, when entering an emergency mode, measurement of SpO2 level, ECG level, and/or additional suitable parameters may be initiated. The staff of MC server 110 or the MC server 110 itself may initiate a call to the patient of wearable device 105, or may send a message etc.

According to some embodiments of the present invention, software or device program updates (referred to herein as “software updates”) may be implemented to enable individualized adaptation of operation parameters of device 105. Customizable software updates may include, for example, customizing one or more modes of operation for each patient, customizing ranges or thresholds for monitoring of an individual patient's parameters, customizing timing of parameter measurements, customizing alert functions, determination of types of measurements to be monitored, customizing diagnostic ranges, adding new features or software improvements, deleting features that are not relevant for a particular patient's condition monitoring, customizing operational modes, correcting software problems, and/or any other suitable modifications. The customized or individualized programs may be programmed directly into wearable device 105 using wire based or wireless data communication, and/or may be remotely transferred to wearable device 105. In this way the timing parameters or other aspects of operation of wearable device 105 may be modified and updated, optionally remotely, by MC server 110, at the discretion of the MC staff or automatically using pre-defined criteria. For example, the MC may remotely initiate a certain mode of operation for wearable device 105, and/or may remotely change vital parameter ranges, etc., optionally for each patient individually. Customized or individualized programs may be programmed directly into a single wearable device 105 or into a group of wearable devices 105.

According to some embodiments of the present invention, measurements of SpO₂ and/or pulse may be executed using reflected light. For example, light may be emitted by a light emitting component, e.g., a light emitting diode (LED) onto a suitable part of a patient's body, and the light reflected from the body may be detected by a light detecting component on wearable device 105. The patient's SpO₂, pulse, blood pressure, SpA₂, and/or other physiological parameters and/or environmental parameters may be calculated from the detected light. The light to be reflected may be emitted in one or more wavelength ranges, as is known in the art. In order to simplify and to clarify, the sub-system or sub-systems for measuring the SpO₂ may be described and illustrated herein as being composed, for example, of an emitting/receiving unit and an electronic unit. In the same manner the sub-system or sub-systems for measuring pulse may be described and illustrated herein as being composed, for example, of an emitting/receiving unit and an electronic unit. Each of the emitting/receiving units may have an emitting light component and a detector to receive the light reflected back to the unit. Such units may also have light filters, electric filter, light amplifiers, electric amplifiers and/or other appropriate elements in the emitting and/or the receiving part. Each of the electronic units may have additional filters and/or amplifiers and/or processing capabilities for running software and/or hardware algorithms in order to extract the values of the SpO₂, pulse, blood pressure, SpA₂, glucose, and/or other physiological parameters.

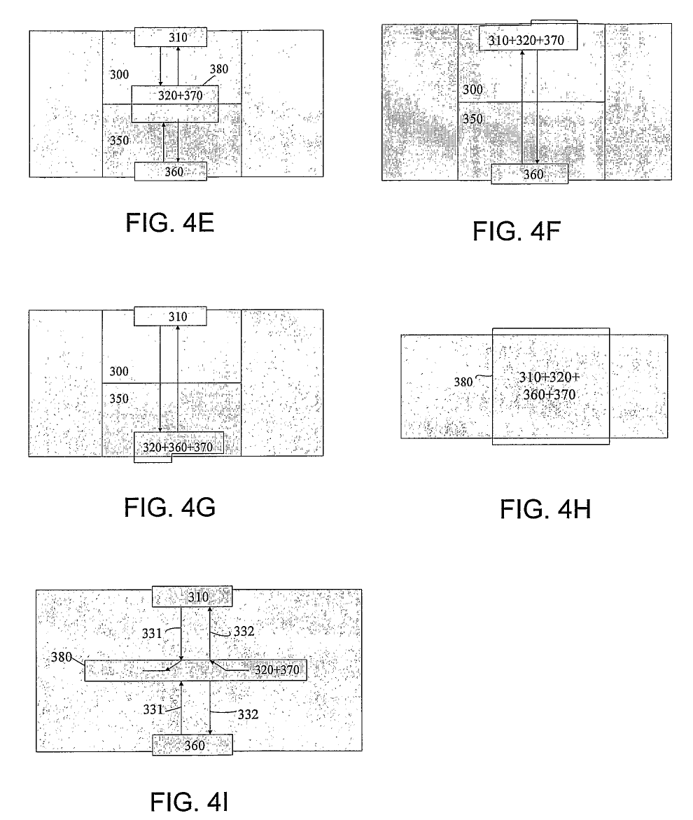

Reference is now made to FIG. 4A, which depicts an emitting/receiving unit 310 and electronic unit 320 of SpO₂ sensor 300, according to an embodiment of the present invention. Units 310 and 320 may be assembled in separate units, for example, printed circuit boards (PCB) or other suitable mounting or etching units, etc., within sensor 300. Similarly, emitting/receiving unit 360 and electronic unit 370 of pulse sensor 350 may be assembled in separate PCBs within sensor 350. In some embodiments a blood pressure sensor, SpA₂ sensor, glucose sensor and/or other suitable sensor may be used, in place of and/or in addition to the SpO₂ sensor and/or pulse sensor.

Reference is now made to FIG. 4B, which depicts emitting/receiving unit 310 and electronic unit 320 assembled within one suitable mounting or etching unit, for example a PCB, within sensor 300, according to an embodiment of the present invention. Emitting/receiving unit 360 and electronic unit 370 may be assembled in separate units, for example separate PCBs, within sensor 350. In some embodiments a blood pressure sensor, SpA₂ sensor, glucose sensor and/or other suitable sensor may be used, in place of and/or in addition to the SpO₂ sensor and/or pulse sensor.

Reference is now made to FIG. 4C, which depicts SpO₂ sensor's emitting/receiving unit 310 and electronic unit 320 assembled in separate units, for example PCBs or other suitable mounting or etching units etc. within sensor 300, according to some embodiments of the present invention. Pulse sensor's emitting/receiving unit 360 and electronic unit 370 may be assembled within one unit, for example a PCB, within sensor 350. In some embodiments a blood pressure sensor, SpA₂ sensor, glucose sensor and/or other suitable sensor may be used, in place of and/or in addition to the SpO₂ sensor and/or pulse sensor.

Reference is now made to FIG. 4D, which depicts SpO₂ sensor's emitting/receiving unit 310 and electronic unit 320 assembled within one suitable mounting or etching unit, for example a PCB, within sensor 300, according to some embodiments of the present invention. Pulse sensor's emitting/receiving unit 360 and electronic unit 370 may be assembled within one unit, for example a PCB, within sensor 350. In some embodiments a blood pressure sensor, SpA₂ sensor, glucose sensor and/or other suitable sensor may be used, in place of and/or in addition to the SpO₂ sensor and/or pulse sensor.

Reference is now made to FIG. 4E, which depicts SpO₂ sensor's emitting/receiving unit 310 assembled within a suitable mounting or etching unit, for example a PCB, within sensor 300, according to some embodiments of the present invention. SpO₂ sensor's electronic unit 320 and pulse sensor's electronic unit 370 may assembled in one unit, for example a PCB, 380 of device 105, and pulse sensor's emitting/receiving unit 360 may be assembled within one unit, for example a PCB, within sensor 350. In some embodiments a blood pressure sensor, SpA₂ sensor, glucose sensor and/or other suitable sensor may be used, in place of and/or in addition to the SpO₂ sensor and/or pulse sensor.

Reference is now made to FIG. 4F, which depicts SpO₂ sensor's emitting/receiving unit 310 and electronic unit 320, and pulse sensor's electronic unit 370 assembled within one suitable mounting or etching unit, for example a PCB, in sensor 300, according to some embodiments of the present invention. Pulse sensor's emitting/receiving unit 360 and may be assembled within one unit, for example a PCB, within sensor 350, according to some embodiments of the present invention. In some embodiments a blood pressure sensor, SpA₂ sensor, glucose sensor and/or other suitable sensor may be used, in place of and/or in addition to the SpO₂ sensor and/or pulse sensor.

Reference is now made to FIG. 4G, which depicts SpO₂ sensor's emitting/receiving unit 310 assembled within a suitable mounting or etching unit, for example a PCB, within sensor 300, according to some embodiments of the present invention. SpO₂ sensor's electronic unit 320 and pulse sensor's emitting/receiving unit 360 and electronic unit 370 may be assembled within one unit, for example a PCB, within sensor 350. In some embodiments a blood pressure sensor, SpA₂ sensor, glucose sensor and/or other suitable sensor may be used, in place of and/or in addition to the SpO₂ sensor and/or pulse sensor.

Reference is now made to FIG. 4H, which depicts SpO₂ sensor's emitting/receiving unit 310 and electronic unit 320, and pulse sensor's emitting/receiving unit 360 and electronic unit 370 assembled within one suitable mounting or etching unit, for example a PCB, within wearable device 105, according to some embodiments of the present invention. In some embodiments a blood pressure sensor, SpA₂ sensor, glucose sensor and/or other suitable sensor may be used, in place of and/or in addition to the SpO₂ sensor and/or pulse sensor.

Reference is now made to FIG. 4I, which depicts Electronic unit 320 and pulse Electronic unit 370 unified within a suitable mounting or etching unit 380, for example a PCB, thereby reducing the number of components, according to some embodiments of the present invention. This reduction is possible because the components of units 320 and 370 may be substantially similar and, therefore, unit 380 may have components that enable operation of both emitting/receiving unit 320 and emitting/receiving unit 360. Such a combined unit 380 may have the capability to execute algorithms and/or methods for extracting a SpO₂ signal from emitting/receiving unit 310, and/or a signal responsive to the pulse from emitting/receiving unit 360. Emitting/receiving unit 310 and Emitting/receiving unit 360 may be assembled in separate units, for example PCBs, within sensor 300 and sensor 350, respectively.

Electrical units 320 and 370 may be combined into single electronic unit 380, which may use a multiple switch to enable sensors 310 and 360 to be operated as desired. In this way, for example, one communication channel 331 and one input/output channel 332 may be used to transmit data from two or more sensors. The communication channel(s) 331 and/or 332 may be, for example, a discrete line, thereby enabling usage of serial communication and/or other suitable methods to control the device sensors. Usage of an electrical switch may enable the power supply to be restricted from the non-used sensor, thereby helping improve the signal received from the active or used sensor. In some embodiments a blood pressure sensor, SpA₂ sensor, glucose sensor and/or other suitable sensor may be used, in place of and/or in addition to the SpO₂ sensor and/or pulse sensor.

Electronic units 320 and 370 may enable driving or operation of sensors 300 and 350, respectively, and reading of information received from sensors 300 and 350 respectively. According to one embodiment, the readings from the sensors may be received via, for example, a switch, e.g., an optical and/or electrical switch. In this way, one measurement may be taken at any time, thereby reducing the number of electronics required, as well as reducing the weight and/or volume of the wearable device 105. In some embodiments, one or more filters and/or amplifiers, and/or other suitable electronics, may be included in at least one sensor channel before being connected to the switch (e.g., between the sensor and the switch). Filters and/or amplifiers, etc., may help improve the quality of the detected signals.

Reference is now made to FIG. 5, which is a schematic illustration of an internal layout of wearable device 105 in accordance with some embodiments of the present invention. Wearable device 105 may include, for example, a main controller 502 to control wearable device operation. Wearable device 105 may include an oxygen level reading controller 506 that may receive input from, for example, one or more SpO₂ transceivers 128. Wearable device 105 may include a pulse reading controller 507 that may receive input from, for example, one or more pulse transceivers 130. Wearable device 105 may generate output signals through main controller 502, and may include other sensors or combinations of sensors.

SpO₂ transceiver 128 and pulse transceiver 130 may include one or more light measurement modules, for example, module 135 and 160, associated with, respectively, SpO₂ transceiver 128 and/or pulse transceiver 130. Light measurement module(s) 135, 160 may include at least one filter. Light measurement module(s) 135, 160 may include at least one amplifier. Light measurement module(s) 135, 160 may include one or more light sensors or optical sensors. Light measurement module(s) 135, 160 may include at least one light emitting diode (LED). Light measurement module(s) 135, 160 may include at least one emitting/receiving unit and at least one electronic unit. In some embodiments the emitting/receiving unit(s) and electronic unit(s) may be assembled in separate etching units, or in a single etching unit, in any combinations. For example, an emitting/receiving unit and an electronic unit of a pulse sensor may be assembled within one etching unit; an emitting/receiving unit and an electronic unit of a SpO₂ sensor may be assembled within one etching unit; an emitting/receiving unit of a pulse sensor and an emitting/receiving unit of a SpO₂ sensor may be assembled within one etching unit; an electronic unit of a pulse sensor and an electronic unit of a SpO₂ sensor may be assembled within one etching unit; an electronic unit of a pulse sensor and an emitting/receiving unit of a SpO₂ sensor may be assembled within one etching unit; an electronic unit of a SpO₂ sensor and an electronic unit of a pulse sensor may be assembled within one etching unit; an electronic unit of a SpO₂ sensor, an emitting/receiving of a pulse sensor, and an emitting/receiving unit of an SpO₂ sensor may be assembled within one etching unit; and an electronic unit of a pulse sensor, an electronic unit of a SpO₂ sensor, and an emitting/receiving unit of a pulse sensor may be assembled within one etching unit. Other suitable combinations may be implemented, for example, wherein elements from additional or alternative measurement modules may be assembled together, in any combination.

Wearable device 105 may include, in place of and/or in addition to the SpO₂ reading controller 506, pulse reading controller 507, and/or blood pressure reading controller 515, additional or alternative controllers 520 (e.g., a blood sugar level reading controller, temperature reading controller, Cardio Impedance (CI) reading controller etc.) that may receive input from one or more suitable sensor(s) or transceivers, and may generate suitable output signals through main controller 502.

In some embodiments, main controller 502 may receive data from input components, for example, data received from functional buttons 112 and 114, emergency buttons 116 and 118, On/Off button 125, and/or from other components, such as service connector 138, charge connector 140, and battery 142. Main controller 502 may generate outputs that may be transferred to output components, for example, display area 134, speaker 136, a modem, an antenna, or any other suitable output devices.

In some embodiments, Oxygen level reading controller 506 may receive signals indicative of vital signs and/or other physiological parameters of the user from sensor 128 and/or 130. Oxygen level reading controller 506 may also receive instruction data, for example via main controller 502, from functionality buttons 112 and 114, emergency buttons 116 and 118, or other suitable sources. Oxygen level reading controller 506 may transfer data, for example via main controller 502, to output components, for example, display area 134, speaker 136, modem, etc.

In some embodiments, Pulse reading controller 507 may receive signals indicative of vital signs and/or other physiological parameters of the user from sensor 129, or other suitable transceivers or sensors. Pulse reading controller 507 may also receive instruction data, for example, via main controller 502, from functionality buttons 112 and 114, emergency buttons 116 and 118, or other suitable sources. Pulse reading controller 507 may transfer data, for example via main controller 502, to output components, for example display area 134, speaker 136, a modem, etc.

In some embodiments, the main controller 502, Oxygen level reading controller 506 and pulse reading controller 507, or additional or alternative controllers, for example, blood pressure controller 515, a blood sugar level controller and/or other suitable controllers 520, may be implemented in a single controller or in multiple separate controllers or any combinations of controllers.

In some embodiments, wearable device 105 may include sensors and controllers to enable measurement and/or processing of, for example, ECG data, blood pressure data, skin temperature data, body temperature data, respiration data, cardio impedance data, blood sugar or glucose level data, and other suitable data. Respective controllers may receive signals indicative of vital signs and/or other physiological and/or environmental parameters of the user from respective sensors. Respective controllers may receive instruction data, for example via main controller 502, from functionality buttons 112 and 114, emergency buttons 116 and 118, or other suitable sources. Respective controllers may transfer data, for example via main controller 502, to various output components, for example display area 134, speaker 136, a communication modem, etc.

In some embodiments of the present invention, Oxygen level reading controller 506 may receive signals from SpO₂ transceiver 128 and/or pulse transceiver 130, and may receive instruction data signals from main controller 502, from function buttons 112 and 114, emergency buttons 116 and 118, etc. Oxygen level reader controller 506 may generate output signals that may be transferred via main controller 502 to one or more output components of wearable device 105 such as display area 134, and to a communication modem to transfer the data regarding measured parameters to MC server 110 and/or to another destination.

In some embodiments of the present invention, data and signals transferred between the components and modules of wearable device 105 may be transferred in, for example, serial communication lines, I/O lines, and/or other suitable designated lines. For example, a V_{BAT} signal may activate an alert indicating that battery 142 is weak, and a V_{CHARGER} signal may activate an alert indicating that battery 142 is charged. Other suitable signals and functions may be implemented.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

What is claimed is:

1. A wireless mobile device for measuring pulse and blood oxygen saturation (SpO₂), the device comprising:
a SpO₂ sensor including a light measurement module to measure SpO₂ levels, said light measurement module using reflected light to measure said SpO₂ levels;
a pulse sensor including a light measurement module to measure pulse levels, said light measurement module using reflected light to measure said pulse levels;
a main controller to receive and process signals from said SpO₂ sensor and from said pulse sensor, and to enable reconfiguration of said SpO₂ and said pulse sensors by commands received from a remote server.

2. The device of claim 1, wherein said light measurement modules include a light emitting diode (LED).

3. The device of claim 1, wherein said light measurement modules include an emitting/receiving unit and an electronic unit.

4. The device of claim 3, wherein said emitting/receiving unit(s) and said electronic unit(s) are assembled in separate etching units.

5. The device of claim 3, wherein said emitting/receiving unit and said electronic unit of said pulse sensor are assembled within one etching unit.

6. The device of claim 3, wherein said emitting/receiving unit and said electronic unit of said SpO₂ sensor are assembled within one etching unit.

7. The device of claim 3, wherein said emitting/receiving units of said pulse sensor and said SpO₂ sensor and said electronic units of said pulse sensor and said SpO₂ sensor are unified within a single etching unit.

8. The device of claim 3, wherein said electronic unit of said pulse sensor and said electronic unit of said SpO₂ sensor are unified within a single etching unit.

9. The device of claim 3, wherein said emitting/receiving unit of said pulse sensor and said emitting/receiving unit of said SpO₂ sensor are unified within a single etching unit.

10. The device of claim 3, wherein said electronic unit of said pulse sensor and said emitting/receiving unit of said pulse sensor and said SpO₂ sensor are unified within a single etching unit.

11. The device of claim 3, wherein said electronic unit of said SpO₂ sensor, said emitting/receiving unit of said pulse sensor, and said emitting/receiving unit of said SpO₂ sensor are unified within a single etching unit.

12. The device of claim 3, wherein said electronic unit of said pulse sensor, said electronic unit of said SpO₂ sensor, and said emitting/receiving unit of said pulse sensor are combined into a single etching unit.

13. The device of claim 3, wherein said electronic unit of said pulse sensor, said electronic unit of said SpO₂ sensor, and said emitting/receiving unit of said SpO₂ sensor are combined into a single etching unit.

14. The device of claim 1, wherein said light measurement module includes a filter.

15. The device of claim 1, wherein said light measurement module includes an amplifier.

16. The device of claim 1, comprising one or more sensors selected from the group consisting of ECG sensors, blood pressure sensors, skin temperature sensors, respiration sensors, perspiration sensors, cardio impedance sensors, and blood sugar level sensors.

17. A system for measuring pulse and blood oxygen saturation (SpO₂), comprising:
a wireless mobile monitoring device, said device including an array of sensors; and
a medical center server enabled to remotely initiate measurements of pulse and blood oxygen saturation (SpO₂) levels of a patient using said monitoring device,
wherein said mobile monitoring device includes a light measurement module, said light measurement model having an emitting/receiving unit and an electronic unit.

18. The system of claim 17, wherein the functioning of said array of sensors may be remotely customized by said server.

19. The system of claim 17, wherein said array of sensors includes at least one physiological sensor and at least one environmental sensor.

20. The system of claim 17, wherein said array of sensors includes at least one physiological sensor.

21. The system of claim 17, wherein said monitoring device is to perform one or more functions selected from the group consisting of measuring parameters, transmitting parameter data, processing parameter data, analyzing parameter data, initiating device actions, updating parameter settings, providing warnings, and providing instructions.

22. The system of claim 17, wherein said monitoring device is to function in one or more of keeper mode, extended mode, and emergency mode.

23. The system of claim 17, wherein said monitoring device is to measure one or more selected parameters continuously and/or intermittently.

24. The system of claim 17, wherein said monitoring device is to automatically send a warning message to said medical center server if parameters measured exceed a selected threshold.

25. The system of claim 17, wherein said remote configuration of said mobile monitoring device includes remotely implementing a customized software update.

26. The system of claim 17, comprising one or more sensors selected from the group consisting of ECG sensors, blood pressure sensors, skin temperature sensors, respiration sensors, perspiration sensors, cardio impedance sensors, and blood sugar level sensors.

27. A method for measuring pulse and blood oxygen saturation (SpO₂), the method comprising transmitting commands to a wireless monitoring device, from a medical center server, to remotely measure pulse and blood oxygen saturation (SpO₂) of a patient using said monitoring device, wherein said mobile monitoring device includes a light measurement module to measure pulse parameters, and a light measurement module to measure SpO₂ parameters.

28. The method of claim 27, comprising transferring measured data to said medical center server.

29. The method of claim 27, comprising remotely initiating one or more actions in said wireless monitoring device, by said medical center server.

30. The method of claim 27, wherein said remote configuration includes implementing customized software updates.

31. The method of claim 27, comprising remotely updating client software in said wireless monitoring device, by said medical center server.

32. A system for remotely measuring physiological and environmental parameters, comprising:
a wireless mobile monitoring device, said device including an array of sensors; and
a medical center server enabled to remotely measure physiological and environmental parameters of a patient using said monitoring device,
wherein said mobile monitoring device includes a light measurement module to measure said physiological parameters, and a light measurement module to measure said environmental parameters.

33. A method for measuring physiological and environmental parameters, the method comprising transmitting commands to a wireless monitoring device, from a medical center server, to remotely measure physiological and environmental parameters of a patient using said monitoring device, wherein said mobile monitoring device includes a light measurement module to measure said physiological parameters, and a light measurement module to measure said environmental parameters.
